(19) United States
(12) Patent Application Publication
(10) Pub. No.: US 2026/0071216 A1
(43) Pub. Date: Mar. 12, 2026

(54) OLIGONUCLEOTIDES FOR MODULATING REGULATORY T CELL MEDIATED IMMUNOSUPPRESSION

(71) Applicant: Turun yliopisto, Turun yliopisto (FI)

(72) Inventors: Riitta Lahesmaa, Turun yliopisto (FI); Ubaid Ullah Kalim, Turun yliopisto (FI); Omid Rasool, Turun yliopisto (FI); Venla Kumpulainen, Turun yliopisto (FI); Syed Bilal Ahmad Andrabi, Turun yliopisto (FI); Senthil Palani, Turun yliopisto (FI)

(21) Appl. No.: 18/873,386

(22) PCT Filed: Jun. 6, 2023
(86) PCT No.: PCT/FI2023/050325

§ 371 Date: Dec. 10, 2024

(30) Foreign Application Priority Data
Jun. 13, 2022 (FI) 20225525

(51) Int. Cl. C12N 15/113; G01N 33/50

(52) U.S. Cl. CPC C12N 15/113; G01N 33/5041; G01N 33/505; C12N 2310/11; C12N 2310/313; C12N 2310/321; C12N 2310/3231; C12N 2310/341

(57) ABSTRACT

The invention relates to a previously uncharacterized gene lincTreg1 and to its long non-coding RNA transcripts as molecular targets for developing novel strategies to combat diseases that involve unbalanced immune system. In particular, the invention relates to oligonucleotides capable of modulating the expression of lincTreg1 for use in treating diseases for which modulating immune response is desirable.

### TECHNICAL FIELD

The present invention relates to functional nucleic acid molecules with gene modulating capabilities. The present invention further relates to oligonucleotides complementary to a previously uncharacterized gene lincTreg1 or its RNA transcript LincTreg1, and to compositions comprising the same. The oligonucleotides are capable of modulating the expression of lincTreg1. Modulation of lincTreg1 expression is beneficial for a range of medical disorders including autoimmunity and cancer.

### BACKGROUND

Regulation of the immune system is critical for controlling immune homeostasis, and imbalance of the immune system can give rise to infections, autoimmune diseases and different forms of cancer. Central to these processes, regulatory T (Treg) cells maintain immune tolerance that distinguish between self and non-self-molecules and balances the immune system by pro- and anti-inflammatory responses.

Commitment and maintenance of Treg lineage is shaped via a complex interplay of several transcription factors (TF) and epigenetic modifiers guiding discrete transcriptional regulatory program. One of the most studied TF is Forkhead box protein 3 (FOXP3) which is associated with Treg cell differentiation and function. However, other TFs (e.g., IKAROS family of TFs, HIC1, NR4A1/2/3) have also been shown to play a significant role in Treg cell differentiation and function. Furthermore, Treg cell specific epigenetic landscape is as important in conferring the regulatory phenotype to Treg cells modulating the immune system.

Epigenetic landscape of a given cell type is, in part, regulated by long noncoding RNAs (lncRNAs), which are usually longer than 200 bp and have no protein-coding capacity. Several lncRNAs have been shown to have crucial function in epigenetic regulation in different cell types. Such LncRNAs can mediate epigenetic modification recruiting chromatin remodelling complex to a specific chromatin to affect various biological processes, including transcriptional regulation, imprinting, and developmental gene expression. However, functions of most lncRNAs are largely unknown.

Owing to their immune modulating function, Treg cells are potential targets for developing novel strategies to combat diseases that involve unbalanced immune system.

### SUMMARY

In one aspect, the invention relates to an oligonucleotide composition for use in treating a disease for which modulating immune response is desirable, the composition comprising or consisting of an oligonucleotide which targets human lincTreg1 gene and specifically binds to a nucleic acid sequence thereof set forth in SEQ ID NO: 1, or to an RNA transcript of the lincTreg1 gene comprising a nucleic acid sequence represented by a nucleic acid sequence selected from the group consisting of SEQ ID NOs: 2-7.

In another aspect, the invention relates to a method for identifying a candidate compound for use in treating a disease for which modulating immune response is desirable. The method comprises the steps of i. contacting induced Treg cells with a test compound, ii. determining whether the test compound affects the expression level of lincTreg1 or LincTreg1 in said cells, and iii. identifying the test compound as a candidate compound for use in treating a disease for which modulating immune response is desirable, if said expression is altered at least by 10%.

Further aspects, embodiments and details are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIG. 1 shows lincTreg1 expression in cells transfected with non-targeting ASOs (NT) or ASOs targeting LincTreg1. The threshold cycle (Ct) values were normalised with the expression of EF1A, which served as an internal control, to generate delta Ct (dCt) values. Less dCt means higher expression. The difference was tested with paired two tailed T test. **** shows p value <0.0001 and ** shows p value <0.01.

FIG. 2 illustrates the expression of FOXP3 at RNA (FIG. 2A) and protein (FIG. 2B) level in cells transfected with non-targeting ASOs (NT) or ASOs targeting LincTreg1. In FIG. 2A, the threshold cycle (Ct) values were normalised with EF1A expression as an internal control. Less dCt means higher expression. In FIG. 2B, median fluorescence intensity (MFI) values have been plotted as obtained in three independent biological replicates. The difference was tested with paired two tailed T test. *** shows p value <0.001 and ** shows p value <0.01 and * shows p value <0.05.

FIG. 3 demonstrates that LincTreg1 regulates immunosuppressive function of iTreg cells. Responder/iTreg ratios are shown at the bottom of the figure. * and ** denote p-value <0.05 and <0.01, respectively, of a two-tailed paired student's T test.

### DETAILED DESCRIPTION

The present invention is based on the identification of a novel gene with a previously unknown function. The gene, denoted herein as lincTreg1, resides on chromosome 6 at 180394-187730 (hg38) and codes for at least six different transcriptional isoforms. In some embodiments, lincTreg1 has a genomic nucleic acid sequence set forth in SEQ ID NO: 1.

Before the invention is further described, it is to be understood that this disclosure is not strictly limited to particular embodiments described herein, as such can of course vary. It is also to be understood that the terminology used herein is for the purpose of describing particular embodiments only, and is not intended to be limiting, since the scope of the present invention will be limited only by the appended claims.

It is also to be noted that, unless defined otherwise, all technical and scientific terms used herein have the same meaning as commonly understood by one of ordinary skill in the art to which this invention belongs.

It is further to be noted that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination.

As used herein and in the appended claims, the singular forms “a”, “an” and “the” mean one or more. Thus, a singular noun, unless otherwise specified, carries also the meaning of the corresponding plural noun. include plural referents unless the context clearly dictates otherwise. As such, the terms “a”, “an”, “one or more” and “at least one” can be used interchangeably. Similarly, the terms “comprising”, “including” and “having” can be used interchangeably.

Turning now to describing the invention and its various embodiments, the newly identified and characterized gene lincTreg1 codes for a long non-coding RNA denoted herein as LincTreg1. Generally, the term “long non-coding RNA” (long ncRNA, lncRNA) as used herein refers broadly to an RNA transcript of more than 200 nucleotides that lacks protein coding potential. Nucleic acid sequences set forth in SEQ ID NOs: 2-7 represent complementary DNA (cDNA) sequences of RNA transcript isoforms 1-6 of lincTreg1, i.e., LincTreg1 isoforms 1-6.

It has now been revealed that lincTreg1 plays an important role in regulatory T cell (Treg) differentiation and function. Moreover, the immune suppressive function of Treg cells can be modulated by targeted regulation of lincTreg1 expression. This opens potential novel therapeutic strategies that modulate lincTreg1 expression in order to combat, mitigate and/or relieve, for example, autoimmunity, infections and different forms of cancer and/or symptoms that arise therefrom.

Generally, the term “regulatory T cell” (Treg cell) as used herein refers to a specialized T cell that can actively suppress activation of the immune system, thereby maintaining homeostasis and self-tolerance. Treg cells exert their immunosuppressive activity, at least, through suppressing cytokine production and proliferation of T effector cells. Treg cells express, at least, the transcription factor Foxp3. Treg cells are primarily generated in the thymus (tTreg), but they may also be generated extrathymically at peripheral sites (pTreg), or induced in cell culture (iTreg) in the presence of cytokines such as transforming growth factor β (TGFβ). Deficiency in the number or function of Treg cells may lead to autoimmunity, whereas an excessive Treg cell response may lead to tumor escape.

In one aspect, the present invention provides lincTreg1 targeting compositions for use in treating a disease for which modulation of immune response is desirable, which compositions comprise or consist of an oligonucleotide capable of specifically targeting and binding to lincTreg1 gene or to an RNA transcript thereof, i.e., LincTreg1, through complementary nucleotides. As a result, the oligonucleotide modulates the expression of lincTreg1 either by silencing or activating lincTreg1 gene expression and/or activity. Accordingly, the lincTreg1 targeting composition may be either a lincTreg1 silencing composition or a lincTreg1 activating composition.

As used herein, the term “transcript” refers broadly to an RNA molecule made in the nucleus by copying a gene's DNA sequence in a transcription process according to principles well known in the art.

The terms “complementarity” and “complementary” are well known in the art and refer to Watson-Crick base pairing where nucleobase adenine (A) in one DNA or RNA strand is represented by nucleobase thymine (T) in its complementary DNA strand or uracil (U) in its complementary RNA strand; whereas nucleobase cytosine (C) in one DNA or RNA strand is represented by nucleobase guanine (G) in its complementary DNA or RNA strand. In other words, the complementary sequence to, for instance, 5′-T-T-C-A-G-3′ is 3′-A-A-G-T-C-5′ or 3′-A-A-G-U-C-5′.

As used herein, the term “targeting” comprises specific binding of an oligonucleotide to a target sequence.

As used herein, the term ““bind” refers to a physical interaction between complementary regions of two single-stranded nucleic acid molecules creating a double-stranded structure by Watson-Crick base pairing.

As used herein, the term “specific binding” or “specifically binding” when made in reference to an oligonucleotide, refers to the discriminatory binding of the oligonucleotide to its target sequence such that oligonucleotide does not substantially cross-react with non-target sequences, i.e., does not have significant off-targeting activity.

Accordingly, oligonucleotides of the invention target and specifically bind to lincTreg1 or to any transcriptional isoform thereof. In some embodiments, the oligonucleotides comprise or consist of a sequence that is complementary to a subsequence of a nucleic acid sequence selected from the group consisting of SEQ ID NOs: 1-7. In some preferred embodiments, the oligonucleotide is designed to be complementary to a region in the 5′-terminal part of lincTreg1 or LincTreg1, such as to a region located within the first 400 base pairs of lincTreg1 or LincTreg1. In some embodiments, the oligonucleotide is complementary to 5 to 60 consecutive nucleotides of a region formed by nucleotides 1-400 of any one of SEQ ID NOs: 1-7. In some embodiments, the oligonucleotide is designed to be complementary to a region of lincTreg1 or LincTreg1 shared by all isoforms thereof.

As readily understood by those skilled in the art, oligonucleotides and oligonucleotide compositions of the invention do not have to be 100% complementary to their target sequences provided that they still specifically bind to the target sequences and silence lincTreg1 without significant off-targeting. Accordingly, oligonucleotides of the invention may in some embodiments comprise or consist of a nucleic acid sequence having at least 80% complementarity, preferably at least 85% complementarity, more preferably at least 90% complementarity, and even more preferably at least 95%, at least 96%, at least 97%, at least 98% or at least 99% complementarity to SEQ ID NOS: 1-7 or a subsequence thereof, provided that the oligonucleotide's ability to silence lincTreg1 gene expression as compared to an oligonucleotide having 100% complementarity to SEQ ID NOs: 1-7 or a subsequence thereof is retained, i.e., is not significantly altered.

The term “% complementarity” as used herein, refers to the number of nucleotides in percent of a contiguous nucleotide sequence in a nucleic acid molecule (e.g., oligonucleotide) which, at a given position, are complementary to (i.e., form Watson Crick base pairs with) a contiguous nucleotide sequence, at a given position of a separate nucleic acid molecule (e.g., the target nucleic acid). The percentage is calculated by counting the number of aligned bases that form pairs between the two sequences, dividing by the total number of nucleotides in the oligonucleotide and multiplying by 100 (i.e., % complementarity=# of identical positions/total # of positions×100).

In some embodiments, the oligonucleotide or the oligonucleotide composition of the invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a subsequence of about 5 to 60, about 10 to 50, or 10 to 40, about 10 to 30, about 10 to 20 or about 20-30 consecutive nucleotides of any one of SEQ ID NOs: 1-7, provided that its specificity to lincTreg1 or LincTreg1 is retained.

Silencing of lincTreg1 can be achieved using various approaches readily available in the art, including for example post-transcriptional silencing through antisense techniques or RNA interference, and targeted gene editing or silencing of transcription through CRISPR-Cas systems.

Multiple antisense techniques are readily available in the art. They all rely on antisense oligonucleotides. Herein, the term “antisense oligonucleotide” (ASO) refers broadly to a short, typically 10-30 nucleotides long, synthetic, single-stranded oligonucleotide that is capable of modulating the expression of a target gene by hybridizing to the gene's RNA transcript, in particular to a subsequence thereof, through complementarity.

In some embodiments, the oligonucleotide is an antisense oligonucleotide. Therefore, the antisense oligonucleotide targets and specifically binds to LincTreg1, and is thereby capable of silencing lincTreg1. In some embodiments, the antisense oligonucleotide targets and specifically binds to LincTreg1 represented by a cDNA sequence set forth in any one of SEQ ID NOs: 2-7, more accurately to a subsequence thereof. Preferably, the subsequence to which the antisense oligonucleotide specifically binds to consists of about 5 to 60, or about 10 to 50, or about 10 to 40, or about 10 to 30 or about 10 to 20 consecutive nucleotides of any one of SEQ ID NOs: 2-7. In other words, the antisense oligonucleotide has or comprises a nucleic acid sequence that is complementary to a subsequence of any one of SEQ ID NOs: 2-7, preferably to a subsequence of about 5 to 60, about 10 to 50, or 10 to 40, about 10 to 30 or about 10 to 20 consecutive nucleotides of SEQ ID NOs: 2-7. As explained above, the antisense oligonucleotide does not have to be 100% complementary to its target sequence, but the complementarity % may vary between 80-100%, provided that the antisense oligonucleotide's lincTreg1 silencing activity remains substantially unaltered.

The chemical composition of the antisense oligonucleotide may be, for example, DNA, RNA, synthetic nucleotide analogs, locked nucleic acid (LNA), peptide nucleic acid (PNA), or it may be composed of mixed polymers containing any number of monomers of DNA, RNA, LNA, PNA, or other nucleic acid analogues.

In cells, DNA: RNA hybrids are substrates for the enzyme RNase H. Thus, DNA-containing antisense oligonucleotides are particularly suitable for silencing lincTreg1 owing to their ability to trigger RNase H-mediated degradation of the target transcript, namely LincTreg1.

In some embodiments, the antisense oligonucleotide has a “gapmer” structure, i.e., is a short antisense oligonucleotide with a DNA segment flacked by segments of RNA mimics. The mimics are typically composed of locked nucleic acids (LNA), 2′-O-Me, 2′-F, or 2′-MOE modified bases. In particular, the LNA and 2′-MOE gapmer modifications have been shown to increase affinity toward target RNA transcripts and endow resistance to nucleases, allowing such molecules to have half-lives between days to several weeks in vivo.

In some embodiments, the antisense oligonucleotide is an LNA antisense oligonucleotide. As used herein, the term “LNA antisense oligonucleotide” refers broadly to an antisense oligonucleotide comprising at least one LNA unit. LNA antisense oligonucleotides can be provided in different formats, including all-LNAs composed exclusively of LNA units and LNA mixmers containing at least one LNA unit and at least one DNA unit. LNA gapmers are the most widely used type of LNA mixmers.

As used herein, the term “locked nucleic acid” (LNA) refers to a nucleic acid analogue in which the ribose ring comprises an extra methylene bridge between the 2′-0 atom and the 4′-C atom. The resulting conformational change facilitates Watson-Crick base paring of the nucleobase component of an LNA with a complementary nucleotide, and increases the stability of the base pairs formed.

In some embodiments, the LNA antisense oligonucleotide is an LNA gapmer. As used herein, the term “LNA gapmer” refers to an antisense oligonucleotide comprising a central DNA stretch of 4 to 12 nucleotides (gap) typically flanked by 1 to 6 residues of LNA nucleotide analogues. In some embodiments, the LNA gapmer comprises a stretch of 4 LNA nucleotide analogues, followed by a stretch of 9 nucleotides, which is followed by another stretch of 4 LNA nucleotide analogues. In some other embodiments, the LNA gapmer comprises a stretch of 3 LNA nucleotide analogues, followed by a DNA stretch of 10 nucleotides, which is followed by another stretch of 3 LNA nucleotide analogues. However, the length of the LNA flanks and that of the central DNA stretch may vary as set forth above.

Non-limiting examples of preferred LincTreg1-specific LNA gapmers include those whose base sequence is as set forth in SEQ ID Nos: 8 and 9. Further preferable LNA gapmers are set forth in SEQ ID NOs: 10-12.

In some embodiments, the antisense oligonucleotide may be an LNA mixmer provided in the form of a headmer or a tailmer, i.e., as an LNA antisense oligonucleotide where one of the LNA flanks is missing. In headmers the 3′ flank is missing, whereas in tailmers the 5′ flank is missing.

In some embodiments, the antisense oligonucleotide is a 2′-MOE gapmer, i.e., a structure wherein a DNA segment is flacked by segments of 2′-MOE units. The number of 2′MOE modifications on both ends of the gapmer may vary as is readily understood by those skilled in the art. 2′MOE gapmers may or may not further comprise phosphorothioate backbone substitutions on some or all of the all nucleotides.

RNA interference (RNAi) is another approach for silencing lincTreg1. As used herein, the term “RNA interference” (RNAi) refers broadly to a biological process in which small RNA molecules can negatively regulate gene expression by directing enzyme complexes to degrade their target RNAs, such as lncRNA transcripts, in a sequence-dependent manner, thereby preventing translation and thus resulting in post-transcriptional gene silencing. Moreover, transcription can be inhibited via the pre-transcriptional silencing mechanism of RNAi, through which an enzyme complex catalyzes DNA methylation at genomic positions complementary to complexed small RNA molecules.

In some embodiments, the lincTreg1 silencing oligonucleotide is a small interfering RNA. As used herein, the term “small interfering RNA” (siRNA) refers to a small double-stranded RNA molecule suitable for RNAi, comprising an antisense strand and a sense strand wherein said antisense strand comprises nucleotide sequence complementary to a subsequence of a target RNA, and the sense strand comprises nucleotide sequence complementary to the said antisense strand. The sense strand and antisense strand can be covalently connected via a linker molecule, which can be a polynucleotide linker or a non-nucleotide linker. The length of the antisense and sense strands may vary and has typically around 21, such as around 19 to 25, nucleotides each.

Non-limiting examples of potential lincTreg1 targeting siRNAs include those comprising or consisting of SEQ ID NO: 13 or 14.

In some embodiments, the lincTreg1 silencing oligonucleotide is a Dicer substrate siRNA. As used herein, the term “Dicer substrate siRNA” (DsiRNA) refers to a somewhat longer double-stranded RNA molecule than the traditional 21-mer siRNA, typically around 25-35 nucleotides in length. DsiRNAs are processed in vivo into active siRNAs by Dicer, and are therefore also suitable for mediating RNAi.

In some embodiments, both the antisense strand and the sense strand of siRNA and DsiRNA molecules may comprise a 3′-terminal overhang of a few, typically 1 to 3 nucleotides. The 3′ overhang may include one or more modified nucleotides, such as a 2′-O-methyl ribonucleotide. The 5′-terminal of the antisense is typically a phosphate group (P). The siRNA and DsiRNA duplexes having terminal phosphate groups (P) are easier to administrate into the cell than a single stranded antisense. In some cases, the 5′-terminal of the sense strand or of both antisense and sense strands may comprise a P group.

In some embodiments, the lincTreg1 silencing oligonucleotide is a short hairpin RNA. As used herein, the term “short hairpin RNA” or “small hairpin RNA” (shRNA) refers to a further class of molecules suitable for inducing RNAi mediated post-transcriptional gene silencing of target genes. The shRNA consist of i) a short nucleotide sequence, typically ranging from 19 to 29 nucleotides, derived from the target gene; ii) a loop, typically ranging between 4 to 23 nucleotides; and iii) a short nucleotide sequence reversely complementary to the initial target sequence, typically ranging from 19 to 29 nucleotides. Dicer processes shRNAs in vivo into different biologically active siRNAs.

Artificial microRNA (amiRNA) precursors are a still further class of small RNAs suitable for mediating RNAi. Artificial miRNA precursors are single-stranded, usually around 21-25 nucleotides in length, and they may have 1 to 3, typically 2, over-hanging 3′ nucleotides.

In cells, an active siRNA associates with a RISC endonuclease complex (RISC=RNA-induced silencing complex). Consequently, the antisense strand is separated from the sense strand, and is targeted to its complementary RNA transcript, such as a lncRNA transcript. Following association of the siRNA with the target, argonaute, a protein from the RISC endonuclease complex, cleaves the target RNA transcript resulting in post-transcriptional silencing of the corresponding target gene. Also amiRNAs and other small single-stranded RNAs (ssRNAs) exert their gene selecting activity through the RISC endonuclease complex.

It is to be understood that also other types of RNAi molecules may be available and they, too, may be employed in accordance with the present invention to silence lincTreg1.

Accordingly, in some embodiments of the present invention, the lincTreg1 targeting composition comprises or consists of an oligonucleotide capable of silencing lincTreg1 through RNAi. The oligonucleotide may be an siRNA, DsiRNA, shRNA or amiRNA molecule, or any other type of small RNA capable of specifically binding to LincTreg1, thereby silencing lincTreg1 through RISC. To this end, the oligonucleotide targets and specifically binds to a nucleic acid sequence selected from SEQ ID NOs: 2-7, in particular a subsequence of any one of SEQ ID NOs: 2-7. Preferably, the subsequence to consists of about 19 to 25, preferably 21, consecutive nucleotides of any one of SEQ ID NOs: 2-7. In other words, the RNAi oligonucleotide has or comprises a nucleic acid sequence that is complementary to a subsequence of any one of SEQ ID NOs: 2-7, preferably to a subsequence of about 19 to 25 consecutive nucleotides of any one of SEQ ID NOs: 2-7. As explained above, the RNAi oligonucleotide does not have to be 100% complementary to its target sequence, but the complementarity % may vary between 80-100%, provided that the RNAi oligonucleotide's lincTreg1 silencing activity remains substantially unaltered. Preferably, the antisense oligonucleotide is complementary to about 19-25, preferably 21 consecutive nucleic acids of any one of SEQ ID NOs: 2-7.

In some embodiments, lincTreg1 may be knocked down either through gene silencing or gene editing by employing a nuclease system comprising at last one genome targeted nuclease and at least one guide RNA comprising at least one targeted genomic sequence. Preferably, the nuclease system is Clustered Regularly Interspaced Short Palindromic Repeats (CRISPR) and CRISPR-associated endonuclease protein (cas) system, i.e., CRISPR-Cas system.

As used herein, the term “guide RNA” (gRNA) molecule refers to a short synthetic nucleic acid molecule that promotes the specific targeting or homing of a gRNA molecule/Cas molecule complex to a target nucleic acid. In other words, gRNA provides both targeting specificity and scaffolding/binding ability for a Cas protein. To this end, gRNA is composed of a “scaffold” sequence necessary for Cas-binding and a user-defined ˜20 nucleotide “targeting domain” which defines the genomic target to be modified. In certain embodiments, the gRNA molecule may be unimolecular of a single RNA molecule (sgRNA). In other embodiments, the gRNA molecule may be a modular gRNA comprising more than one, and typically two, separate RNA molecules.

In some embodiments, the Cas protein is a Cas9 protein. The CRISPR-Cas9 system can be configured to inactivate the lincTreg1 gene, for example, through introduction of a stop codon, a frameshift mutation, or a larger deletion into the genomic lincTreg1 DNA using means and methods readily available in the art.

In some embodiments, the Cas protein is a catalytically dead Cas9 protein (dCas9). When fused to a Krüppel-associated box (KRAB) repression domain it can site-specifically silence transcription, an approach known as CRISPR interference (CRISPRi). This allows repression of the lincTreg1 locus without editing the genome, thus avoiding unintentional deletion of active regulatory elements.

In some embodiments, the Cas protein is Cas13 protein. When provided with gRNA complementary to the target RNA, CRISPR-Cas13 can efficiently cleave the RNA target. It is therefore envisaged that the CRISPR-Cas13 system is particularly suitable for knocking down lncRNAs, such as LincTreg1, in mammalian cells.

In some embodiments, a modification of the CRISPR-Cas system may be employed to activate transcription, an approach known as CRISPR activation (CRISPRa). In such embodiments, dCas9 is fused with a transcriptional activator, such as VP6, VP16, VP64 or VPR, to increase target gene expression.

Accordingly, in some embodiments of the present invention, the lincTreg1 targeting composition comprises or consists of a CRISPR-Cas system capable of silencing lincTreg1 through gene editing or CRISPRi. To this end, the CRISPR-Cas system comprises a gRNA, such as an sgRNA, having a targeting domain that is complementary to a target sequence in the genomic lincTreg1. The targeting domain comprises a nucleotide sequence that is e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% complementary to the target sequence on the target nucleic acid, with the proviso explained above. For systems such as CRISPR-Cas9 and CRISPR-dCas9, the target nucleic acid is the genomic lincTreg1, i.e., SEQ ID NO: 1; whereas for the CRISPR-Cas13 system, the target nucleic acid is the LincTreg1 transcript represented by any one of cDNA sequences set forth in SEQ ID NOs: 2-7. In some embodiments, the targeting domain may be 10 to 30 nucleotides in length. Some or all of the nucleotides of the targeting domain can be a chemically modified oligonucleotide, including any and all modifications mentioned herein.

Non-limiting examples of potential lincTreg1 targeting gRNAs include those comprising or consisting of SEQ ID NOs: 15 to 18.

Normal, unmodified oligonucleotides have low stability under physiological conditions because of their degradation by nucleases present in the living cell or biological fluid. Thus, although in some embodiments oligonucleotides of the invention, such as antisense oligonucleotides, RNAi molecules, and oligonucleotides for CRISPR-Cas9 systems, may be unmodified, preferred embodiments concern corresponding chemically modified oligonucleotides, especially those with enhanced stability against chemical and enzymatic degradation and/or with increased affinity toward the target nucleic acid, namely those represented by any one of SEQ ID NOs: 1-7. The chemical modification may involve any part of the nucleotide, i.e., the phosphodiester linkage (the backbone of DNA and RNA), the ribose sugar and/or the nucleobase.

Non-limiting examples of chemically modified phosphodiester linkages include those wherein one or more oxygen atoms are replaced by sulfur, amino, alkyl or alkoxy groups. Especially preferred are oligonucleotides where some or all of the inter-nucleotide phosphodiester linkages are replaced by phosphorothioate linkages. Further backbone modifications are well known in the art and include, for example, boranophosphate linkage modifications.

Non-limiting examples of numerous ribose modifications available, and aimed at improving affinity and/or nuclease resistance, include those wherein substituent groups other than hydrogen are introduced into one or more ribose rings in the nucleic acid molecule at the 2′, 3′, 4′ or 5′ positions. Preferred modifications include 2′ substitutions, including but not limited to substitutions wherein the ribose 2′-H or 2′-OH group is replaced with alkyl, alkenyl, alkoxy, allyl, alkoxyalkyl, halo, amino, azido or sulfhydryl groups. Particularly preferred substitutions include 2′-deoxy, 2′-O-methyl, 2′-methoxyethyl (MOE), and 2′-fluoro substitutions to name some.

Sugar modifications also include modifications where the sugar moiety is replaced with a non-sugar moiety, such as in the case of peptide nucleic acids (PNA), or morpholino nucleic acids. Locked nucleic acids (LNA) represent one preferable ribose sugar modification.

Peptide nucleic acids (PNAs) are oligonucleotide analogues in which the sugar-phosphate backbone has been replaced by a pseudopeptide skeleton. They bind DNA and RNA with high specificity and selectivity through Watson-Crick base pairing. The resulting PNA-RNA and PNA-DNA hybrids have high thermal stability and resistance to proteases and nucleases, making them ideal constituents of targeting oligonucleotides of the invention.

Non-limiting examples of nucleobase modifications suitable for modifying the oligonucleotides of the invention include replacing the purine or pyrimidine with a modified purine or pyrimidine, such as substituted purine or substituted pyrimidine. Suitable modified nucleobases include, for example, isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

Oligonucleotides useful in the present invention can be designed and analyzed by using commercial or non-commercial algorithm programs available in the art. This may be achieved, for example, by loading the full-length cDNA sequence of lincTreg1 to an algorithm program. In one embodiment, the nucleic acid sequence set forth in any one of SEQ ID NOs: 2-7 represents the cDNA sequence of lincTreg1. Algorithm-generated lincTreg1 targeting sequences can then be screened, e.g., trough genome wide DNA sequence alignment (BLAST), to minimize or predict total off-target activity across the genome. In other words, each possible lincTreg1 targeting sequence can be ranked according to its total predicted off-target cleavage. The top-ranked sequences represent those that are likely to have the greatest on-target and the least off-target cleavage. Candidate molecules can then be synthetized, and validated in vitro and/or in vivo according to methods available in the art.

Different algorithm programs can be used for designing and analyzing different types of gene silencing oligonucleotides. These computer programs sieve out the given target sequence with an appropriate set of rules, depending on the type of gene silencing molecule to be designed. For example, algorithm programs suitable for designing and analyzing siRNAs include Eurofins MWG Operon's Online Design Tool and a stand-alone program developed by Cuia et al; whereas gRNA targeting domain sequences can be designed and analyzed, for example, using a software tool available at http://crispr.mit.edu/.

In accordance with the above, lincTreg1 targeting compositions comprise or consist of oligonucleotides capable of modulating lincTreg1 expression either by specifically binding to lincTreg1 or LincTreg1. In some embodiments the oligonucleotide of the invention is capable of modulating the expression of lincTreg1 by inhibiting or down-regulating it. In some other embodiments the oligonucleotide of the invention is capable of modulating the expression of lincTreg1 by activating or up-regulating it. Preferably, such modulation produces a modulation (either inhibition or activation as the case may be) of expression of at least 20% compared to the normal expression level of lincTreg1, more preferably at least 30%, 40%, 50%, 60%, 70%, 80%, or 90% modulation compared to the normal expression level of the target.

The target modulation is triggered by the hybridization between a contiguous nucleotide sequence of the oligonucleotide and the target nucleic acid. In some embodiments the oligonucleotide of the invention comprises mismatches between the oligonucleotide and the target nucleic acid. Despite mismatches hybridization to the target nucleic acid may still be sufficient to show a desired modulation of lincTreg1 expression. Reduced binding affinity resulting from mismatches may advantageously be compensated by increased number of nucleotides in the oligonucleotide and/or an increased number of modified nucleosides capable of increasing the binding affinity to the target, such as 2′ modified nucleosides, including LNA and 2′-MOE, present within the oligonucleotide sequence.

The present lincTreg1 targeting compositions comprising or consisting of oligonucleotides that modulate lincTreg1 expression are capable of modulating the immunosuppressive activity of Treg cells. In some embodiments the composition is capable of increasing the immunosuppressive activity of Treg cells, while in some other embodiments the composition is capable of decreasing the immunosuppressive activity of Treg cells. Preferably, such modulation produces a modulation (either increase or decrease as the case may be) of the immunosuppressive activity of Treg cells of at least 20% compared to the normal immunosuppressive activity of Treg cells, more preferably at least 30%, 40%, 50%, 60%, 70%, 80%, or 90% modulation compared to the normal immunosuppressive activity of Treg cells.

Since Treg cells exert their immunosuppressive activity, at least partly, through suppressing cytokine production and proliferation of T effector cells, in vitro assays that involve determination of cytokine production and/or proliferation of T effector cells in the presence of Treg cells and a lincTreg1 targeting oligonucleotide may be employed for assessing the lincTreg1 targeting oligonucleotide's capability of modulating immunosuppression. In vitro assays suitable for this purpose are readily available in the art.

lincTreg1 targeting oligonucleotides of the invention can be synthetized by means and methods readily available in the art.

Delivery of lincTreg1 targeting oligonucleotides can be accomplished in two principally different ways: 1) endogenous transcription of a nucleic acid sequence encoding the oligonucleotide, where the nucleic acid sequence is located in an expression construct, or 2) exogenous delivery of the oligonucleotide.

For endogenous transcription, lincTreg1 targeting oligonucleotides may be inserted into suitable expression systems using methods known in the art. Non-limiting examples of such expression systems include retroviral vectors, adenoviral vectors, lentiviral vectors, other viral vectors, expression cassettes, and plasmids, such as those encapsulated in pegylated immunoliposomes (PILs), with or without one or more inducible promoters known in the art. If double stranded RNA, such as siRNA or DsiRNA, is employed, both RNA strands may be expressed from a single expression construct from the same or separate promoters, or the strands may be expressed from separate expression constructs.

For exogenous delivery, lincTreg1 targeting oligonucleotides are typically complexed with liposome or lipid-based carriers, cholesterol conjugates, or polyethyleneimine (PEI) in different sizes and shapes. Suitable routes of administration for exogenous delivery, with or without said complexing, include, but are not limited to, parenteral delivery, enteral delivery, local administration, and topical administration as known to a person skilled in the art.

In one aspect, the invention provides pharmaceutical compositions comprising any of the aforementioned lincTreg1 targeting oligonucleotides and/or oligonucleotide complexes or salts thereof and a pharmaceutically acceptable carrier. As used herein, the term “pharmaceutically acceptable carrier” refers broadly to a carrier substance or diluent with which the oligonucleotide or the oligonucleotide complex is combined to facilitate administration and that is physiologically acceptable to the recipient. The selected carrier should not abrogate the biological activity and properties of the oligonucleotide or the oligonucleotide complex but minimize any degradation of thereof as wells as minimize adverse side effects in the recipient.

Formulation and composition of the pharmaceutical composition depends on different variables, such as the intended route of administration and the dose to be administered, as is known to those skilled in the art. In some embodiments, the oligonucleotide is used in the pharmaceutically acceptable diluent at a concentration ranging, for example, from 4 μg/ml to 40 mg/ml.

Parenteral administration of the pharmaceutical composition, if used, is generally applied by injection, for example intravenously, intraperitoneally, subcutaneously, or intramuscularly. Preparations for parenteral administration are typically sterile aqueous or non-aqueous solutions, suspensions or emulsions, but the preparation may also be provided in a concentrated form or in a form of a powder to be reconstituted on demand. Slow release or sustained release formulation are also contemplated. Means and methods for formulating preparations for parenteral administration are readily available in the art, and those skilled in the art can easily select appropriate physiologically suitable carriers, adjuvants and/or excipients depending on the desired specifics of the preparation.

Non-limiting examples of aqueous carriers for parenteral and other pharmaceutical preparations include sterile water, water-alcohol solutions, saline, and buffered solutions at physiological pH. Parenteral vehicles include sodium chloride solution, Ringer's dextrose solution, dextrose plus sodium chloride solution, Ringer's solution containing lactose, or fixed oils. Intravenous vehicles include fluid and nutrient replenishers, electrolyte replenishers, such as those based on Ringer's dextrose solution, and the like.

Non-limiting examples of non-aqueous carriers for parenteral and other pharmaceutical preparations include solvents such as propylene glycol, polyethylene glycol, vegetable oils such as olive oil, animal-based oils such as fish oils, shark oils, injectable organic esters such as ethyl oleate, and any combinations thereof.

If the parenteral preparation is provided as a concentrated solution or dispersion, or as a powder, aqueous or non-aqueous carriers mentioned above may be used for reconstitution. A solution for the reconstitution may be provided in the same package as the concentrate or powder. If lyophilization is used for preparing the powder, it may be beneficial to use cryoprotectants including, without limitation, polymers (e.g. povidones, polyethylene glycol, dextran), sugars (e.g. sucrose, glucose, lactose), amino acids (e.g. glycine, arginine, glutamic acid) and albumin, and any combinations thereof.

Enteral administration of the pharmaceutical composition, if used, may be applied, for example, through oral administration. Compositions for oral administration include, without limitation powders, granules, capsules, sachets, tablets and aqueous or non-aqueous solutions and suspensions, and any combinations thereof. Means and methods for formulating preparations for enteral administration are readily available in the art, and those skilled in the art can easily select appropriate physiologically suitable carriers, adjuvants and/or excipients depending on the desired specifics of the preparation.

Enteral administration of the pharmaceutical composition, if used, may be applied, for example, through inhalable administration. Compositions for inhalable administration include, without limitation powders, granules, carriers, particle and aqueous or non-aqueous solutions and suspensions or combination thereof. Means and methods for formulating preparations for enteral administration are readily available in the art, and those skilled in the art can easily select appropriate physiologically suitable carriers, adjuvants and/or excipients depending on the desired specifics of the preparation.

Topical administration of the pharmaceutical composition, if used, may be applied, for example, through transdermal administration, transmucosal administration, epicutaneous administration, intranasal administration, and administration by an inhalant. Depending on the administration route, formulations for topical administration can include ointments, lotions, creams, gels, drops, suppositories, sprays, liquids, powders and slow release or sustained release formulations or solid objects, and any combinations thereof. Means and methods for formulating preparations for topical administration are readily available in the art, and those skilled in the art can easily select appropriate physiologically suitable carriers, adjuvants and/or excipients depending on the desired specifics of the preparation.

Amounts and regimens for administration of a pharmaceutical composition disclosed herein can be determined readily by those with ordinary skill in the clinical art of treating autoimmune diseases and cancer. Generally, dosing will vary depending on considerations such as: age, gender and general health of the subject to be treated; kind of concurrent treatment, if any; frequency of treatment and nature of the effect desired; severity and type of disease or condition in question; causative agent of the disease and other variables to be adjusted by the individual physician. A desired dose can be administered in one or more applications to obtain the desired results. For example, the pharmaceutical composition may be administered in a single daily dose, or the total daily dosage may be administered in divided doses of e.g. two, three or four times daily. The pharmaceutical composition may be provided, for example, in unit dosage forms or in extended release formulations.

Because Treg cells critically contribute to suppression of immune responses, their therapeutic depletion or functional inactivation may activate the immune system and enhance immune responses in the body. It is envisaged that the same can be achieved by blocking the immunosuppressive activity of Treg cells through silencing of lincTreg1.

Accordingly, the present invention provides lincTreg1 silencing oligonucleotides and compositions for use in activating the immune system and/or stimulating a therapeutic immune response in the treatment of a disease for which activation of the immune system and/or stimulating the therapeutic immune response is desirable. This aspect of the invention can also be formulated as a method of activating the immune system in a subject in need thereof. The method comprises inhibiting the immunosuppressive activity of Treg cells by administering a therapeutically efficient amount of a lincTreg1 silencing composition to the subject, wherein inhibiting the immunosuppressive activity of the Treg cells activates the immune system and/or stimulates a therapeutic immune response in the subject.

As used herein, the term “treatment” or “treating” refers to the administration of the lincTreg1 targeting composition to a subject for purposes which include not only complete cure of a disease, but also alleviation and amelioration of a disease or symptoms related thereto.

As used herein, the term “therapeutically efficient amount” refers to an amount by which harmful effects of a disease or condition are, at a minimum, ameliorated.

As used herein, the term “subject” refers to an animal subject, preferably to a mammalian subject, more preferably to a human subject. Herein, the term “patient” refers to a human subject.

As used herein, the term “immune response” refers to a reaction which occurs within a subject for the purpose of defending itself against substances it sees as harmful or foreign. In an immune response, the immune system recognizes foreign antigens (usually proteins) on the surface of such substances and attacks and destroys, or tries to destroy, them. Cancer cells also have antigens on their surface. Sometimes, the immune system sees these antigens as foreign and mounts an immune response against them, helping the body fight cancer. Autoimmunity is defined as an immune response toward a self-antigen, i.e. any molecule that is a normal body constituent of the subject. A transplanted organ may also incite an immune response when it is identified as non-self.

As used herein, the term “immunosuppression” refers to a reduction of the activation or efficacy of the immune system. In general, deliberately induced immunosuppression is performed to prevent the body from rejecting an organ or tissue transplant, or for the treatment of autoimmune diseases such as type I diabetes, multiple sclerosis, rheumatoid arthritis, Crohn's disease, systemic lupus erythematosus, psoriasis and Sjögren's syndrome. Accordingly, the term “immunosuppressive” refers to an ability of an entity to prevent the immune system from reacting to antigens completely or partly, for example in order to prevent autoimmunity or transplanted organs from being rejected. In the context of the present invention, “immunosuppressive” refers in particular to Treg cells' natural ability to suppress immune response in a subject.

Immunosuppression may increase immune tolerance. As used herein, the terms “immune tolerance” and “immunotolerance” are interchangeable and refer broadly to a state of an active, carefully regulated unresponsiveness of the immune system to substances or tissues that have the capacity to elicit an immune response in a given organism. In other words, immunotolerance refers to the prevention of an immune response against a particular antigen. For instance, the immune system is generally tolerant of self-antigens, so it does not usually attack the body's own cells, tissues, and organs. However, when tolerance is lost, disorders like autoimmune disease or allergy may occur.

Cancer immunotherapy aims to promote tumor elimination through the activation of innate and adaptive immune responses. Treg cells critically contribute to the occurrence and persistence of tumor-induced tolerance and are the dominant immune escape mechanism in early tumor progression. Therefore, therapeutic depletion or functional inactivation of Treg cells may improve responses to cancer immunotherapy.

Thus, in some embodiments, lincTreg1 silencing compositions are provided for use in improving cancer immunotherapy in a subject, or for treating a subject having cancer. The former aspect can be formulated as a method of improving cancer immunotherapy in a subject in need thereof. The method comprises decreasing the immunosuppressive activity of Treg cells by administering a therapeutically efficient amount of a lincTreg1 silencing composition to the subject, wherein decreasing the immunosuppressive activity of the Treg cells improves the cancer immunotherapy in the subject.

In some further embodiments, lincTreg1 silencing compositions are provided for use in suppressing immune tolerance to cancer antigens in a subject in need thereof. This aspect can be formulated as a method of suppressing immune tolerance to a cancer antigen in the subject. The method comprises decreasing the immunosuppressive activity of Treg cells by administering a therapeutically efficient amount of a lincTreg1 silencing composition to the subject, wherein decreasing the immunosuppressive activity of the Treg cells reduces the immune tolerance of cancer antigens in the patient. Reduced immune tolerance to a cancer antigen may help the body combat cancer.

More generally, some embodiments provide lincTreg1 silencing compositions for use in suppressing immune tolerance in a subject having a disease for which suppressing immune tolerance is desirable, such as cancer. This aspect can also be formulated as a method of suppressing immune tolerance in a subject in need thereof. The method comprises decreasing the immunosuppressive activity of Treg cells by administering a therapeutically efficient amount of a composition comprising a lincTreg1 silencing oligonucleotide or complex thereof to the subject, wherein decreasing the activity of the Treg cells reduces the immune tolerance in the subject.

On the other hand, it is envisaged that therapeutic activation of Treg cells may inactivate the immune system and dampen or reduce immune responses in the body. In accordance with the present invention, this may be achieved by increasing the immunosuppressive activity of Treg cells by activation of lincTreg1.

Accordingly, the present invention provides lincTreg1 activating compositions for use in treating a disease for which reducing immune response is desirable. Accordingly, in one aspect, the present invention provides lincTreg1 activating compositions for use in reducing immune response. This aspect of the invention can be formulated as a method of inactivating the immune system in a subject in need thereof. The method comprises increasing the immunosuppressive activity of Treg cells by administering a therapeutically efficient amount of a lincTreg1 activating composition to the subject, wherein increasing the immunosuppressive activity of the Treg cells inactivates and/or dampens the immune system in the subject.

In some embodiments, lincTreg1 activating compositions are provided for use in increasing immune tolerance, or for treating a disease for which increasing immune tolerance is desirable. This aspect can also be formulated as a method of increasing immune tolerance in a subject in need thereof. The method comprises increasing the immunosuppressive activity of Treg cells by administering a therapeutically efficient amount a lincTreg1 activating composition to the subject, wherein increasing the activity of the Treg cells increases the immune tolerance in the subject. Preferably, the increased immune tolerance is directed to one or more disease antigens.

In some embodiments, lincTreg1 activating compositions are provided for use in increasing immune tolerance to self-antigens in a subject, or for treating an autoimmune disease or for preventing allograft rejection following tissue transplantation. This aspect can also be formulated as a method of increasing immune tolerance to a self-antigen in a subject in need thereof, or as a method of treating an autoimmune disease or of preventing graft rejection in a subject. The method comprises increasing the immunosuppressive activity of Treg cells by administering a therapeutically efficient amount of a lincTreg1 activating composition to the subject, wherein increasing the activity of the Treg cells increases the immune tolerance of a self-antigen in the subject or is effective in treating the autoimmune disease or for preventing the allograft rejection in the subject. Non-limiting examples of autoimmune disease to be treated include type I diabetes, multiple sclerosis, rheumatoid arthritis, Crohn's disease, systemic lupus erythematosus, psoriasis and Sjögren's syndrome.

Without being limited to any mechanism of action, Forkhead box protein 3 (Foxp3), a transcription factor only expressed in the Treg cell lineage, not only contributes to a distinct genetic signature to Treg cells, but is also crucial for Treg cell differentiation and function. Indeed, reduction of the Foxp3 expression has been reported to be indicative of the suppression of immune tolerance to the cancer antigens in the patient.

Importantly, silencing of lincTreg1 results in concomitant decrease in the expression of Foxp3, supporting the role of lincTreg1 in the controlling the immunosuppressive activity of Treg cells. Moreover, transcriptome of LincTreg1 deficient iTreg cells revealed a general loss of Treg signature gene (FOXP3, CTLA4, PDCD1, IL21R, IKZF4, CD79A) expression with concomitant increase in the expression of effector T cell signatures (GBP4, LRRN3, IL13, CYP1B1, TNFSF10, MX2, PLSCR1, ANXA1, and EVI2B). This result is in accordance with the general knowledge that while some lncRNA regulate gene expression in cis, having transcriptional enhancer-like function for genes on the same chromosome, other lncRNA loci function in trans, producing a lncRNA transcript that functions at locations genetically unlinked and spatially distant from their site of production. Notably, LincTreg1 acts in trans.

Accordingly, in some embodiments, the oligonucleotide composition of the invention provided for use in various purposes such as for use in treating cancer or autoimmunity, activating the immune system, stimulating a therapeutic immune response, improving cancer immunotherapy, suppressing immune tolerance to cancer antigens, inactivating the immune system, dampening an immune response, increasing immune tolerance to self-antigens through interaction with the lincTreg1, LincTreg1 and/or Foxp3.

The present invention also provides a screening method for identifying small molecule modulators of lincTreg1 and/or LincTreg1. Such modulators may serve as candidate compounds for drug development to obtain agents for use in treating diseases for which modulating immune response is desirable, including agents that modulate immunosuppressive capacity of Treg for different purposes such as for activating the immune system, stimulating a therapeutic immune response, improving cancer immunotherapy, suppressing immune tolerance to cancer antigens and/or treating cancer. Some embodiments provide a method for identifying a candidate compound for treating a disease for which modulating immune response is desirable, including a method for identifying a candidate compound for modulating immunosuppressive activity of Treg cells, the method comprising: i) contacting iTreg cells with a test compound, ii) determining whether the test compound modulates the expression of lincTreg or LincTreg1, for example by using TqMan qPCR assay, and iii) identifying the test compound as a candidate compound for use in treating said disease, preferably through modulating immunosuppressive activity of Treg cells, if the expression of lincTreg1 or LincTreg1 is altered, for example by at least 10%, preferably by at least 20%, more preferably by at least 30%, 40%, 50%, 60%, 70%, 80%, 90% or more.

Means and methods for obtaining iTreg cells are available in the art. The method of identifying said candidate compounds may also include determining the effect of the test compound on the expression of FOXP3. Moreover, the candidate compound's capability to modulate immunosuppressive activity of Treg cells may be verified by means and methods available in the art, including the assay disclosed in Example 4.

### EXPERIMENTAL PART

### Example 1. Identification and Characterization of lincTreg1 Gene Structure

During an earlier transcriptome analysis of human iTreg cells, the inventors observed a locus specifically expressed in iTreg cells as compared to activated T cells (Th0). The locus was overlapping the 3′ region of the lncRNA gene LOC285766 (RP3-416J7.4/AL035696.3) which is transcribed from the anti-sense (reverse/−) strand of chromosome 6. The inventors suspected that the mapped reads may not come from LOC285766 because: (A) the reads were present only in the 3′region, (B) the junctions supported by the mapped reads were not present in the LOC285766 gene and (C) the exon-intron junctions on the LOC285766 gene were not supported by the mapped reads. Thus, based on the read junctions found in the RNA-seq data, the inventors assembled on the sense (Forward/+) strand six isoforms of the gene which we propose to name as lincTreg1. The genomic nucleic acid sequence of lincTreg1 is set forth in SEQ ID NOs: 1.

To determine the boundaries of the gene and the exon-intron junctions, the inventors performed a series of random amplification of cDNA ends (RACE)-PCR and regular PCR reactions combined with sanger sequencing. Based on results from these analyses, the inventors identified the gene with the beginning at chr6:180394 (hg38) and the end at chr6: 187730 (hg38).

To confirm if the reads in the RNA-seq are indeed coming from the sense strand, the inventors analysed H3K4me3 promoter mark using CUT&Tag-seq on 72 h differentiated iTreg and Th0 cells. A clear promoter mark near the 5′ end of lincTreg1 was found, and the promoter mark was specific to iTreg cells. Importantly, the promoter for LOC285766, the overlapping gene transcribed from the anti-sense strand, was neither accessible in T cells nor showed any H3K4me3 mark, suggesting that lincTreg1 (LOC105374869) but not LOC285766 is expressed in iTreg cells. The FANTOM5 data also showed a promoter near lincTreg1 transcription start site in T cells.

Further, we confirmed lincTreg1 expression using Pacific biosciences (PacBio) based Iso-seq. Iso-seq analysis confirmed that lincTreg1 is transcribed from the sense strand, and that one of the isoforms of the transcript contains four exons and has 2393 bases. The northern blot analysis further confirmed a specific band of approximately 2.5 kb suggesting the expression of the transcript in iTreg cells. Taken together, based on RNA-seq data, the inventors identified a novel iTreg-specific lincTreg1 gene with six isoforms of which expression of isoforms 6 was confirmed by PCR, PacBio sequencing and Northern blotting. Complementary DNA sequences of the six transcriptional LincTreg1 isoforms are set forth in SEQ ID NOs: 2-7

### Example 2. Validation of Selected Antisense Oligonucleotides (ASOs) for Silencing LincTreg1 Expression

### Materials and Methods

### Antisense Oligonucleotides

LincTreg1-targeting, especially LincTreg1 isoform 6-targeting, LNA gapmer oligonucleotides were ordered from Exiqon, later acquired by Qiagen. The LNA gapmer oligonucleotides included those having base sequences set forth below. Positions of the LNA modifications are not shown.

Design ID: 512016-2:
(SEQ ID NO: 8)
5′-GTCACGAGCTCTTAAG-3′
Design ID 624672-3:
(SEQ ID NO: 9)
5′-GTAGGTAGAATGGTCT-3′

Additional LincTreg1 targeting ASOs, namely phosphorothioate backbone substituted gapmer structures with either ‘locked nucleic acid’ (LNA) or 2′-O-methoxyethylribose (2′-MOE) chemical modifications were synthetized by Integrated DNA Technologies (IDT).

A first set of additional ASOs contained 16 nucleotide long target sequences with phosphorothioate backbone substitutions (*) on all nucleotides and LNA modifications (+) at the first three and last three nucleotides of 5′ and 3′ends, respectively. This type of additional ASOs included the following:

(SEQ ID NO: 10)
5′-+G*+T*+A*G*G*T*A*G*A*A*T*G*G*+T*+C*+T-3′
(SEQ ID NO: 11)
5′-+G*+T*+C*A*C*G*A*G*C*T*C*T*T*+A*+A*+G-3′
(SEQ ID NO: 12)
5′-+G*+T*+A*G*T*G*A*G*T* G*T*C*A*+C*+G*+A-3′

CD4+ Cell Isolation and Differentiation to iTreg Cells

Mononuclear cells were isolated from the cord blood of healthy neonates at Turku University Central Hospital using Ficoll-Paque PLUS (GE Healthcare, Fairfield). CD4+ T cells were then isolated using a bead-based positive isolation kit (Invitrogen, Cat #11331D). CD25 depletion was performed using LD columns (Miltenyi Biotec GmbH, Cat #130-092-983C). CD4+CD25-cells from multiple donors (three or more) were activated directly or pooled before activation with plate-bound anti-CD3 (500 ng/24-well culture plate well; Beckman Coulter, cat #IM-1304) and soluble anti-CD28 (500 ng/ml; Beckman Coulter, cat #IM1376) at a density of 2× 106 cells/mL of X-vivo 15 serum-free medium (Lonza). For iTreg differentiation, the medium was supplemented with IL-2 (12 ng/ml), TGF-β (10 ng/ml) (both from R&D Systems), all-trans retinoic acid (ATRA) (10 nM) (Sigma-Aldrich), and human serum (10%) and cultured at 37° C. in 5% CO2. Control Th0 cells were stimulated with plate-bound anti-CD3 soluble anti-CD28 X-vivo 15 serum-free medium without cytokines.

### LincTreg1 Gene Knockdown

Freshly isolated CD4+CD25-cells were suspended in Optimem I (Invitrogen) and transfected with ASOs using the nucleofection technique (Lonza). Four million cells were transfected with 300 pmol of control ASO (NT) or ASOs targeting LincTreg1. The transfected cells were allowed to rest for 24 h in RPMI 1640 medium (Sigma-Aldrich) supplemented with pen/strep, 2 mM L-glutamine and 10% FCS at 370 C (2×106 cells/ml) and subsequently activated and cultured as described above.

### RNA Isolation and TaqMan Assay

RNA was isolated (RNeasy Mini Kit, QIAGEN, Cat #74106) and treated in-column with DNase (RNase-Free DNase Set; QIAGEN, Cat #79254) for 15 min. The removal of genomic DNA was ascertained by an additional treatment of the samples with DNase I

(Invitrogen, Cat #18068-015). RNA was quantified using Nanodrop 2000 and quality was measured using BioRad Experion and Agilent Bioanalyzer. All the samples had good RNA quality. 100 ng RNA was used to synthesize cDNA using Invitrogen's SuperScript™ Il Reverse Transcriptase kit (ThermoFisher, cat #18064-014). Either Universal ProbeLibrary System (Roche Life Science), used to design the primers and probes, or TaqMan RT PCR assays from Applied Biosystems/ThermoFisher was used to measure gene expression. Dilutions of 1:5 or 1:10 cDNA were used for the qRT-PCR assays in QuantStudio™ 12K Flex Real-Time PCR System (Thermofisher). The qRT-PCR data analysis was performed using Quantstudio software. Data was normalized by substracting cycle threshold (CT) value of the internal control, which is EF1A in these assays, from the CT value of the target gene.

### Results

Successful LincTreg1 Knockdown with all Tested ASOs

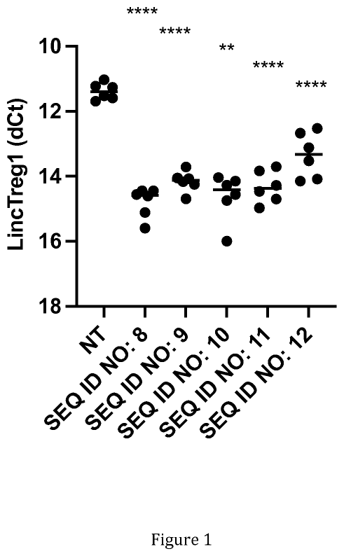

TaqMan analysis of cells transfected with either LincTreg1-specific ASOs or non-targeting ASO (5′-AACACGTCTATACGC-3′; SEQ ID NO: 19; NT) indicated that the most significant LincTreg1 knockdowns were obtained with the LNA-gapmer-ASOs described above, namely those of SEQ ID NO: 8, SEQ ID NO: 9, SEQ ID NO: 10, SEQ ID NO: 11, and SEQ ID NO:12 (FIG. 1).

### Example 3. Impact of LincTreg1 Silencing on FOXP3 Expression

### Methods

### Intracellular Staining and Flow Cytometry

Intracellular staining was performed with buffer sets of Human Regulatory T Cell Staining Kit (eBioscience/ThermoFisher Scientific, Cat #88-8999-40), following the manufacturer's protocol. The following antibodies were used: anti-human FOXP3-PE (eBioscience, Cat #12-4776-42) and rat IgG2a isotype control (eBioscience, Cat #72-4321-77A). Samples were acquired on BD LSR Fortessa analyzer (BD Biosciences, Franklin Lakes, NJ) and analysed with FlowJo (FLOWJO, LLC).

### Results

### Significant FOXP3 Reduction Induced by the Most Efficient New LincTreg1 LNA-Gapmer-ASOs

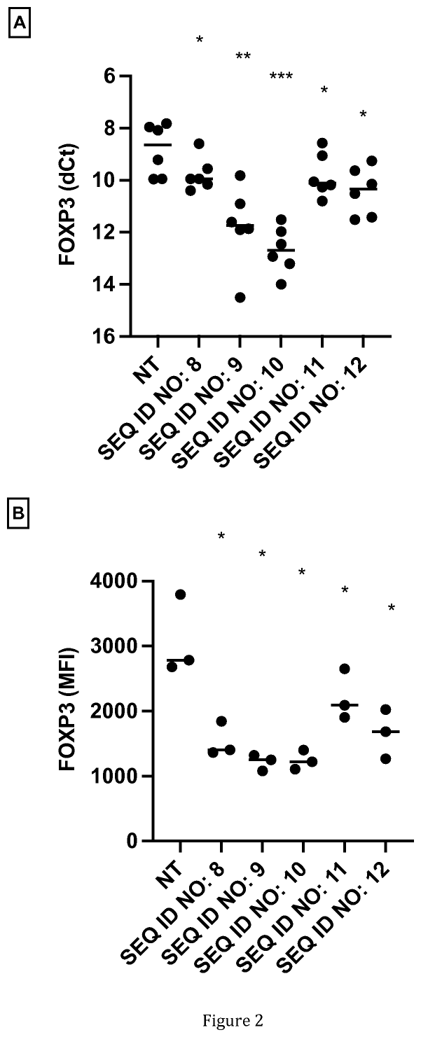

As illustrated in FIG. 2, cells transfected with those gapmer-ASOs which most efficiently knocked down LincTreg1 showed significantly reduced FOXP3 expression both on RNA (A) and protein level (B). The above-disclosed LNA gapmers of SEQ ID NO: 8, SEQ ID NO: 9, ISEQ ID NO: 10, SEQ ID NO. 11, and SEQ ID NO: 12 resulted in the most prominent FOXP3 reduction.

### Example 4. Impact of LincTreg1 Silencing on Immunosuppressive Activity of Treg Cells

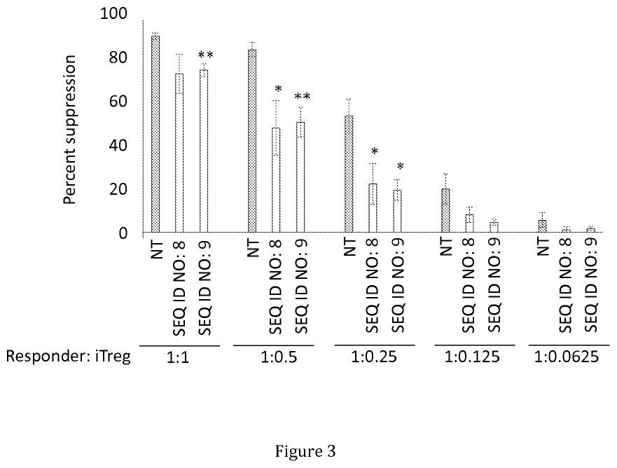

The inventors measured suppressive function of LincTreg1 silenced iTreg cells using in vitro suppression assays, where LincTreg1 deficient iTreg cells were activated and co-cultured in with CTV labelled CD4⁺T cells isolated from peripheral blood (responder cells) at different responder/iTreg ratios. The proliferation of responder cells, as measured by CTV dye dilution, was quantified after 72 h of activation. The percentage suppression was calculated using the following formula: % suppression=[% of dividing cells (Tres−iTreg)/% of dividing cells in Tres]×100. The data was plotted (mean+/−SEM) from five independent experiments. The results shown in FIG. 3 demonstrate that silencing of lincTreg1 results in decreased immunosuppressive activity of Treg cells.

1. An oligonucleotide composition for use in treating a disease for which modulating immune response is desirable, wherein the composition comprising an oligonucleotide which targets human lincTreg1 gene and specifically binds to a nucleic acid sequence thereof set forth in SEQ ID NO: 1, or to an RNA transcript of the lincTreg1 gene comprising a nucleic acid sequence represented by a nucleic acid sequence selected from the group consisting of SEQ ID NOs: 2-7.

2. The oligonucleotide composition for use according to claim 1, wherein the oligonucleotide specifically binds to 10 to 30 consecutive nucleotides of any one of SEQ ID NOs: 1-7, or has a nucleic acid sequence which is at least 80% complementary to 5 to 60 consecutive nucleotides of any one of SEQ ID NOs: 1-7 without losing lincTreg1 specificity.

3. The oligonucleotide composition for use according to claim 1, wherein the oligonucleotide modulates immunosuppressive capacity of regulatory (Treg) cells.

4. The oligonucleotide composition for use according to claim 1, wherein the oligonucleotide modulates the expression of Forkhead box protein 3 (Foxp3).

5. The oligonucleotide composition for use according to claim 1, wherein the oligonucleotide silences lincTreg1 expression.

6. The oligonucleotide composition for use according to claim 5, wherein the oligonucleotide is an antisense oligonucleotide, a small interfering RNA (siRNA) oligonucleotide, a Dicer substrate siRNA (DsiRNA) oligonucleotide, a short hairpin RNA (shRNA) oligonucleotide, an artificial microRNA (amiRNA) oligonucleotide or a guide RNA (gRNA) oligonucleotide for a CRISPR-Cas system.

7. The oligonucleotide composition for use according to claim 1, wherein the oligonucleotide activates lincTreg1 expression.

8. The oligonucleotide composition for use according to claim 7, wherein the oligonucleotide is a guide RNA (gRNA) oligonucleotide for a CRISPR-Casd9 system.

9. The oligonucleotide composition for use according to claim 1, wherein the treatment is for activating the immune system, stimulating a therapeutic immune response, improving cancer immunotherapy, suppressing immune tolerance to cancer antigens and/or treating cancer.

10. The oligonucleotide composition for use according to claim 1, wherein the treatment is for inactivating the immune system, dampening an immune response, increasing immune tolerance to self-antigens and/or treating an autoimmune disease.

11. The oligonucleotide composition for use according to claim 1, further comprising a pharmaceutically acceptable carrier.

12. The oligonucleotide composition for use according to claim 1, wherein the composition is intended for parenteral delivery, enteral delivery, local administration, or topical administration.

13. A method for identifying a candidate compound for use in treating a disease for which modulating immune response in desirable, the method comprising:
i. contacting induced Treg cells with a test compound,
ii. determining whether the test compound affects the expression level of lincTreg1 or LincTreg1 in said cells, and
iii. identifying the test compound as a candidate compound for use in treating a disease for which modulating immune response is desirable, if said expression is altered at least by 10%.

14. The method according to claim 13, wherein the test compound is an oligonucleotide composition comprising an oligonucleotide which targets human lincTreg1 gene and specifically binds to a nucleic acid sequence thereof set forth in SEO ID NO: 1, or to an RNA transcript of the lincTreg1 gene comprising a nucleic acid sequence represented by a nucleic acid sequence selected from the group consisting of SEO ID NOs: 2-7.


SEQUENCE LISTING (19 sequences)

SEQ ID NO: 1 - DNA, 7537 bp, synthetic construct
  source 1..7537: mol_type = other DNA
gtcaaaacggaccaatcagctctctgtaaaatgggccaatcagcaggatgtgggtggggtcagataagggaataaaagcgggctgcggaggcaaccgtggtaagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaattccagacacaagaccatctcatctcaagagaccaggacgtccgggcacccctagaatgtagctgctggcagagcctgaagtctatccagctcatgctcttccctgatatccttgacagattgtgggatggttcagcctctgcaaggacatggccaatgatggggggctggttttgggggcagcactctaggcaagggaagaaataaagtgtctgggaactggcgtggtgaaggggcactttgtggagaagatgtgttcacatttggatgtgtcgagggtggaatgatatgaggaaagtcaaagggtgcccacccccagagcctaggtgtgggcacagcactgcaccacgccttaggggagattggactctgggcagagatcccaagagaagcaggagttggagtcttgggggccagtgtgatagggaactaactttactttaaagccaatttgtcagtaagaaaaaacgaggtgcaccttgtaggagaaaaagtcacaagcttggtgtgcttgaagtcacaagccttgctgtgcttggtgagagggatcccgagtgtacttgaggcactctcctgtttaaaaggggaacggtggttttcttttctcagtgtaggttagtgtaaaaaatctgctttgtaattgcataaggtctcagcacaaacaaaatcacagaaaatgtgaaatgtattataacagtcaacttgcttagaatgtagttgaacctgcatgcagtattgagacaccagacaactgttttcagtctccacatttctttcatttcctttatgaaagttacattaagaaaaaaaataactcccttgagtattctgatattttcggtgagtgaaacagtaatctcagcaaagttcaatcttggtatcagacagaatttcaggttgtttcatctaattgccacttaataccaaagacatgatgtttattagcatgtatttctaatgagaaactattatagtcgtcctcaattgtctgaaatatgaagcggaaaaagaaaattatgagccccagataccagaccagttgctgatcagatagaatatcgtggttccagcatattatgaaataaaaattcaaagactgaataccatgcaaaaggtcaatattcggcagcatctgagaagtcatacatctagatagacctaaactataagtgcagaagtgctacagtaaactatatgggctttctactaagtactggctaattttaaaacatactgaaatttatgtacattagtctttgttataagaattgagaatccgcattgtattataaagttgatctacaacagtcactttattataaaatattttcggcctggcgcgttggctcacacctgtaatcccagcactttgtgaggccgaggcaggcagatcacctgaggttgggagttcaagaccagcctgaccaacatggagaaaaccccatctctactaaaaatacaaaattagccgggcgtggaggcaggtgcctgtaatcccagctactccggaggctgaggcaagagaatcgcttgaacctgggaggcggaggttgcagtgagccaagatctcaccattgcactccagcctgggcaacaagagtgaaactccatctcaaaaaaaaaaaaaaaaaaaaattcaccagaaggaaagtgcttacctatttgaacaactctgtacgggtctatacagtattccaaggctcttcaataagcacatggtgatgacccaggtcagtcgtggcaaggttggggaattagaacaaacctctgtggcaaacacagattatagcttgaagtgagcatcacggatttctacaacaaacactctgacaaatatttcctgtgataaacatccacgttctcgaggtgggacaattaacccaaactttccaaattacataaaaacttttgctctatctaattaagctgataaattcattattttaaatgacaacatacaaaagaagtatagatttttacttaaaacgtattggatatgtgggtattctcttgtctttctaagcaaatgctaatatcagttcagtgcttaacaatgtcagcagcaactgggccctctaagtctcctgctactgaagtttgagggatcaagcctcaccgtaattttgacaaagactaaattcaagatgtctgaggcttacaattttcttattctacttaatgaggtagagaagttcatttggaattgagcactccattagactgtttcagctaaatttcaggtggcattttctaacaataagagctgttgatgctttggaaggcaacaggtttgcttcactggaagtgatgatggccttccagaaagggtgaggcaaggttttctaagatctcatccaactaaatgattcagaaagcgccagcctggggattccaggtgcttgtgactcggggatctttctgccatcccacctcacctgttagtcaccttttgaaattgggtatgattgaaatggagtttagggaatgctgtaataatatatctcttgcttgcctaattcacagaaacgtgcacaggcaaactgtgatgctaagtgggggctttgatcctttgatgccgggccctcctcccacccggagccatacctatttctcccttgcccgttctcctgcctctgcgttcagatgtgcttcaggcctgagacaggaggaacctatcaatgaaggaggaaatgttccccagctcctcaaacacgaggcctccttctgcaactcaactttctgctcccaggacaggataatctgaagaccccaccgcttcacctcatctgaagactcagttaaagaggagcgggcaggcagctgggggattcattctctgggagtgggagcagaagggagccccttgtcttgtttttgttccttcccagttgacccacagctggagtgcgggctgggcaagtaggtgctgggcaacttctggggagactcaggcctccttattggccatcacaaagccccagagaaaaagatgcttccaaaaactgcaagataagaggaacaggtaaaggtcatgggaggattaagtagagcacagcaggctgcttcacgaaccctctgtggccttccccaggtgggccagcatggcagctgctaacattgagtctaggccatcaggcaaggtgaattagggacagtaccagtggggaaagtacctcggtgtagcccatgcactgtctgccacccacccatgaagttcaacctcttctagtgctcttttctgttttcatggtcctggagtgacttgacctcatttagtcattctatgattatcccatttacaggcaaggagcatttttattgttgttaatgctcttaactctagcaagctatgaatttaggacgtgtctgtgccctttggcaaacttactgtctccttaggaattttgtgcccgatttcttttgctcttccagactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtaggtaagagtggtacaagcttccctgtcacatgggtaggagtcaacttccctttcctttgtggtgctaaaccccccgccacacctcgagtaaacagggccctttctaagcccagctcctgtcttgtctgggcacagcatgtgttttctcctgggaccctgaagcacacatccattctactcatgtaaaacagacagctgcataagagaaaataatatattcacgtgtacagctgttggcaagaagtcttcagagatggagaaaccgatgaagaatcttgctgcaagatcccaacctatgccaagggccactgcgagaagccagcatggacacgaaattcacacccagggcagctcctgtgtcttctgagccttctcatgagggctgcaagcacagtggtctggtactgcctgcctgtaatgttttcttttagaatagaccctgtgtgtagatgaaagagcattcgacatcattactacgtgggatgctgagaaccaggtgttttcttctctgggcataagctcctgaggctcccgaaagtctcatcctcctgggccgaggatgctgaagggagcagtgggatggaaggcgggcccagaaagcacacgggcaggcagaatctgctctgcccagctccagtgtcttgtctttactgtgctcaccgtatgttttcagaaagacaggccaagaacaaaagtgggagccacctaaatgtccaaccatgatggtccatgaacacagtggaatgtgcaagataattgtgaaaattgtgcggaagtatggggaactgcttaggatacacagttaagtgaataaagcagcatgttaagtgttatatatgccatggttacaattccaaaaatatgtatgtacataggcaaattctggaagaataaaaccagaagaaaatgttgtattaaagtgatagcatcatggatgtatatagatagcctttgatgtctttgttggaagaggccatccttgcaccctgtcccttgctgagcatgccacaactgcaaggccctgagtgtcccttagctggcccatttctcaggtccagctgataactctgagaggtgaggtaacattgacccctccccatgatcctccaggcaaacagcaggctggcttctggcttgctgtaagcacagcgccttctccaagctccacgcttcacgctgcacacacggcattcatctgagcctgctgtgtcacctgtgagggaactgggactgaggggaactgacacagaagtgcggctggacataacatccaggaaacagtagcaggttgactgaatagcttctaagaaggataaaatcaaatcccagatgacagtcttaataaggttgcgataccaatttgaaattacagttgactcttaaacaccggtttgaactgcgagggtccacttagagatggatttttttttccaaaaaatacagtccgtgggttttgtatccacaaccaaacacagatagaaaatatagtattctcaggatttaaatctgcccgtacggaagcctggcttttcataaccttcggtccccaggactcactatgggacttgagtatgtgcagattttcatgtcttacccatggccctggaagcaatttcctacagattcccagggatgactgtatttgcctaatgtttctttcccagcagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatacctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttacgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttgcatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaatctcactttcgctgttctctgggtctctgagtccattcttcgggtttggatgggtgagtttgtttcttacgcctagtgagggactgggccaggcagtctct

SEQ ID NO: 2 - DNA, 4766 bp, Synthetic construct
  source 1..4766: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaattccagacacaagaccatctcatctcaagagaccaggacgtccgggcacccctagaatgtagctgctggcagagcctgaagtctatccagctcatgctcttccctgatatccttgacagattgtgggatggttcagcctctgcaaggacatggccaatgatggggggctggttttgggggcagcactctaggcaagggaagaaataaagtgtctgggaactggcgtggtgaaggggcactttgtggagaagatgtgttcacatttggatgtgtcgagggtggaatgatatgaggaaagtcaaagggtgcccacccccagagcctaggtgtgggcacagcactgcaccacgccttaggggagattggactctgggcagagatcccaagagaagcaggagttggagtcttgggggccagtgtgatagggaactaactttactttaaagccaatttgtcagtaagaaaaaacgaggtgcaccttgtaggagaaaaagtcacaagcttggtgtgcttgaagtcacaagccttgctgtgcttggtgagagggatcccgagtgtacttgaggcactctcctgtttaaaaggggaacggtggttttcttttctcagtgtaggttagtgtaaaaaatctgctttgtaattgcataaggtctcagcacaaacaaaatcacagaaaatgtgaaatgtattataacagtcaacttgcttagaatgtagttgaacctgcatgcagtattgagacaccagacaactgttttcagtctccacatttctttcatttcctttatgaaagttacattaagaaaaaaaataactcccttgagtattctgatattttcggtgagtgaaacagtaatctcagcaaagttcaatcttggtatcagacagaatttcaggttgtttcatctaattgccacttaataccaaagacatgatgtttattagcatgtatttctaatgagaaactattatagtcgtcctcaattgtctgaaatatgaagcggaaaaagaaaattatgagccccagataccagaccagttgctgatcagatagaatatcgtggttccagcatattatgaaataaaaattcaaagactgaataccatgcaaaaggtcaatattcggcagcatctgagaagtcatacatctagatagacctaaactataagtgcagaagtgctacagtaaactatatgggctttctactaagtactggctaattttaaaacatactgaaatttatatacattagtctttgttataagaattgagaatccgcattgtattataaagttgatctacaacagtcactttattataaaatattttcggcctggcgcgttggctcacacctgtaatcccagcactttgtgaggccgaggcaggcagatcacctgaggttgggagttcaagaccagcctgaccaacatggagaaaaccccatctctactaaaaatacaaaattagccgggcgtggaggcaggtgcctgtaatcccagctactccggaggctgaggcaagagaatcgcttgaacctgggaggcggaggttgcagtgagccaagatctcaccattgcactccagcctgggcacagagtgaactcatctcaaaaaaaaaaaaaaaaattcaccagaaggaaagtgcttacctatttgaacaactctgtacgggtctatacagtattccaaggctcttcaataagcacatggtgatgacccaggtcagtcgtggcaaggttggggaattagaacaaacctctgtggcaaacacagattatagcttgaagtgagcatcacggatttctacaacaaacactctgacaaatatttcctgtgataaacatccacgttctcgagactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcatgtgttttctcctgggaccctgaagcacacatccattctactcatgtaaaacagacagctgcataagagaaaataatatattcacgtgtacagctgttggcaagaagtcttcagagatggagaaaccgatgaagaatcttgctgcaagatcccaacctatgccaagggccactgcgagaagccagcatggacacgaaattcacacccagggcagctcctgtgtcttctgagccttctcatgaggtctgcaagcacagtggtctggtactgcctgcctgtaatgttttcttttagaatagaccctgtgtgtagatgaaagagcattcgacatcattactacgtgggatgctgagaaccaggtgttttcttctctgggcataagctcctgaggctcccgaaagtctcatcctcctgggccgaggatgctgaagggagcagtgggatggaaggcgggcccagaaagcacacgggcaggcagaatctgctctgcccagctccagtgtcttgtctttactgtgctcaccgtatgttttcagaaagacaggccaagaacaaaagtgggagccacctaaatgtccaaccatgatggtccatgaacacagtggaatgtgcaagataattgtgaaaattgtgcggaagtatggggaactgcttaggatacacacagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatacctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttacgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttgcatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 3 - DNA, 4100 bp, Synthetic construct
  source 1..4100: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaattccagacacaagaccatctcatctcaagagaccaggacgtccgggcacccctagaatgtagctgctggcagagcctgaagtctatccagctcatgctcttccctgatatccttgacagattgtgggatggttcagcctctgcaaggacatggccaatgatggggggctggttttgggggcagcactctaggcaagggaagaaataaagtgtctgggaactggcgtggtgaaggggcactttgtggagaagatgtgttcacatttggatgtgtcgagggtggaatgatatgaggaaagtcaaagggtgcccacccccagagcctaggtgtgggcacagcactgcaccacgccttaggggagattggactctgggcagagatcccaagagaagcaggagttggagtcttgggggccagtgtgatagggaactaactttactttaaagccaatttgtcagtaagaaaaaacgaggtgcaccttgtaggagaaaaagtcacaagcttggtgtgcttgaagtcacaagccttgctgtgcttggtgagagggatcccgagtgtacttgaggcactctcctgtttaaaaggggaacggtggttttcttttctcagtgtaggttagtgtaaaaaatctgctttgtaattgcataaggtctcagcacaaacaaaatcacagaaaatgtgaaatgtattataacagtcaacttgcttagaatgtagttgaacctgcatgcagtattgagacaccagacaactgttttcagtctccacatttctttcatttcctttatgaaagttacattaagaaaaaaaataactcccttgagtattctgatattttcggtgagtgaaacagtaatctcagcaaagttcaatcttggtatcagacagaatttcaggttgtttcatctaattgccacttaataccaaagacatgatgtttattagcatgtatttctaatgagaaactattatagtcgtcctcaattgtctgaaatatgaagcggaaaaagaaaattatgagccccagataccagaccagttgctgatcagatagaatatcgtggttccagcatattatgaaataaaaattcaaagactgaataccatgcaaaaggtcaatattcggcagcatctgagaagtcatacatctagatagacctaaactataagtgcagaagtgctacagtaaactatatgggctttctactaagtactggctaattttaaaacatactgaaatttatatacattagtctttgttataagaattgagaatccgcattgtattataaagttgatctacaacagtcactttattataaaatattttcggcctggcgcgttggctcacacctgtaatcccagcactttgtgaggccgaggcaggcagatcacctgaggttgggagttcaagaccagcctgaccaacatggagaaaaccccatctctactaaaaatacaaaattagccgggcgtggaggcaggtgcctgtaatcccagctactccggaggctgaggcaagagaatcgcttgaacctgggaggcggaggttgcagtgagccaagatctcaccattgcactccagcctgggcacagagtgaactcatctcaaaaaaaaaaaaaaaaattcaccagaaggaaagtgcttacctatttgaacaactctgtacgggtctatacagtattccaaggctcttcaataagcacatggtgatgacccaggtcagtcgtggcaaggttggggaattagaacaaacctctgtggcaaacacagattatagcttgaagtgagcatcacggatttctacaacaaacactctgacaaatatttcctgtgataaacatccacgttctcgagactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatccctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttatgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttccatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 4 - DNA, 3747 bp, Synthetic construct
  source 1..3747: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaattccagacacaagaccatctcatctcaagagaccaggacgtccaggcacccctagaatgtagctgctggcagagcctgaagtctatccagctcatgctcttccctgatatccttgacagattgtgggatggttcagcctctgcaaggacatggccaatgatggggggctggttttgggggcagcactctaggcaagggaagaaataaagtgtctgggaactggcgtggtgaaggggcactttgtggagaagatgtgttcacatttggatgtgtcgagggtggaatgatatgaggaaagtcaaagggtgcccacccccagagcctaggtgtgggcacagcactgcaccacgccttaggggagattggactctgggcagagatcccaagagaagcaggagttggagtcttgggggccagtgtgatagggaactaactttactttaaagccaatttgtcagtaagaaaaaacgaggtgcaccttgtaggagaaaaagtcacaagcttggtgtgcttgaagtcacaagccttgctgtgcttggtgagagggatcccgagtgtacttgaggcactctcctgtttaaaaggggaacggtggttttcttttctcagtgtaggttagtgtaaaaaatctgctttgtaattgcataaggtctcagcacaaacaaaatcacagaaaatgtgaaatgtattataacagtcaacttgcttagaatgtagttgaacctgcatgcagtattgagacaccagacaactgttttcagtctccacatttctttcatttcctttatgaaagttacattaagaaaaaaaataactccttgagtattctgatattttcgactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcatgtgttttctcctgggaccctgaagcacacatccattctactcatgtaaaacagacagctgcataagagaaaataatatattcacgtgtacagctgttggcaagaagtcttcagagatggagaaaccgatgaagaatcttgctgcaagatcccaacctatgccaagggccactgcaaggagccagcatggacacgaaattcacacccagggcagctcctgtgtcttctgagccttctcatgagggctgcaagcacagtggtctggtactgcctgcctgtaatgttttcttttagaatagaccctgtgtgtagatgaaagagcattcgacatcattactacgtgggatgctgagaaccaggtgttttcttctctgggcataagctcctgaggctcccgaaagtctcatcctcctgggccgaggatgctgaagggagcagtgggatggaaggcgggcccagaaagcacacgggcaggcagaatctgctctgcccagctccagtgtcttgtctttactgtgctcaccgtatgttttcagaaagacaggccaagaacaaaagtgggagccacctaaatgtccaaccatgatggtccatgaacacagtggaatgtgcaagataattgtgaaaattgtgcggaagtatggggaactgcttaggatacacacagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatccctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttatgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttccatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 5 - DNA, 3081 bp, Synthetic construct
  source 1..3081: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaagtccagacacaagaccatctcatctcaagagaccaggacgtccaggcacccctagaatgtagctgctggcagagcctgaagtctatccagctcatgctcttccctgatatccttgacagattgtgggatggttcagcctctgcaaggacatggccaatgatggggggctggttttgggggcagcactctaggcaagggaagaaataaagtgtctgggaactggcgtggtgaaggggcactttgtggagaagatgtgttcacatttggatgtgtcgagggtggaatgatatgaggaaagtcaaagggtgcccacccccagagcctaggtgtgggcacagcactgcaccacgccttaggggagattggactctgggcagagatcccaagagaagcaggagttggagtcttgggggccagtgtgatagggaactaactttactttaaagccaatttgtcagtaagaaaaaacgaggtgcaccttgtaggagaaaaagtcacaagcttggtgtgcttgaagtcacaagccttgctgtgcttggtgagagggatcccgagtgtacttgaggcactctcctgtttaaaaggggaacggtggttttcttttctcagtgtaggttagtgtaaaaaatctgctttgtaattgcataaggtctcagcacaaacaaaatcacagaaaatgtgaaatgtattataacagtcaacttgcttagaatgtagttgaacctgcatgcagtattgagacaccagacaactgttttcagtctccacatttctttcatttcctttatgaaagttacattaagaaaaaaaataactccttgagtattctgatattttcgactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatccctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttatgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttccatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 6 - DNA, 3057 bp, Synthetic construct
  source 1..3057: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaagtccagacacaagaccatctcatctcaagagaccaggacgtccaggcacccctagaatgtagctgctggcagagcctgaagtctatccagctcatgctcttccctgattaagaaaaaaaataactcccttgagtattctgatattttcgactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcatgtgttttctcctgggaccctgaagcacacatccattctactcatgtaaaacagacagctgcataagagaaaataatatattcacgtgtacagctgttggcaagaagtcttcagagatggagaaaccgatgaagaatcttgctgcaagatcccaacctatgccaagggccactgcgagaagccagcatggacacgaaattcacacccagggcagctcctgtgtcttctgagccttctcatgaggtctgcaagcacagtggtctggtactgcctgcctgtaatgttttcttttagaatagaccctgtgtgtagatgaaagagcattcgacatcattactacgtgggatgctgagaaccaggtgttttcttctctgggcataagctcctgaggctcccgaaagtctcatcctcctgggccgaggatgctgaagggagcagtgggatggaaggcgggcccagaaagcacacgggcaggcagaatctgctctgcccagctccagtgtcttgtctttactgtgctcaccgtatgttttcagaaagacaggccaagaacaaaagtgggagccacctaaatgtccaaccatgatggtccatgaacacagtggaatgtgcaagataattgtgaaaattgtgcggaagtatggggaactgcttaggatacacacagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatacctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttacgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttgcatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 7 - DNA, 2391 bp, Synthetic construct
  source 1..2391: mol_type = other DNA
taagctgtgcttgcatcatgtttggaaactgtcgtactgcgcgaagctttgcagcttccctccttaaaggcagtgagatggcaaacccactgggaggaatgagaaactccaggcgcaccaccttaagagctcgtgacactcactacaaaggtctgcagccactcctgaagcaagcgagaccacaaacccaccagcagaaagaaactgaacacgtccaaacatctgaaggaactagctccagacacaccatctttaaaaactgtcacactcactgtgaaggtctgcggctttattcttttagtcagtgagaccaagtccagacacaagaccatctcatctcaagagaccaggacgtccaggcacccctagaatgtagctgctggcagagcctgaagtctatccagctcatgctcttccctgattaagaaaaaaaataactcccttgagtattctgatattttcgactgctgcccatgctcctctgccctgccgagcactctgggagatggacctggttggacactagagctccaccctgcagcttccaggtgggctcagtagatggagatcctggaggagactgtagagcagctgctggcacagttgtcctggagctacctgggcccagatgccccagctctctccatgcagaccattctacctacctctcctctcatactgggctctccgtgtagcagactcatgtctgtcttgtgcactgctgtgttcctggcaccatgggggagcgtggtgcagagcaagtttcctcagatggccctttgtggatggactacaggagcagagatgtgcaagaagcaagtttcagaaggaattcaaaccaaaatgtagagtttgaaggcaacagtgacctttgcaatcatccctccctgttattcagtaacaaagccaccaccatgtatcaagcacccacaggccacacctgtcttctggacgattttcggggcatcatatttcaatgttctgtccatttccaggcacaaagtgctttcactcacattcggtccttgctgcaactgcaagccactgaagacaggtgtgtctgatcccaacgtggagcccacagtaacagcaagcagcgactgttgcttgtgcaggaggccctgtgtgccacatgttgctcttcactactgatcagggctgtttacgtgggtcccatcacagctgctctgtgactagggaacttgacgtctccatttttcaactgaaaacacagaagtacaaaggggttcaccaccttgtccacggttgccctgcttctaactggtcatgttggaataaaaacaagcaactgtcatccgggatattaaccacgcaggctgtggtgttgcaaagcaggggagcacggagcagggagcataccaccctgctgagatcagctcactcacaagtggcagggctagggctgcagtgcagctcacttatgggcactgtgtaagcctgaaagcaaagtccacatccaaaggtcgtagtggccccaaaacagcttcctgacctgcttctgtggtcccagacctcttgctgctgaggtaccttagctttccaattactcttgccaattctgcctgcccagtactgctaaaaggccacttccctccgtcccatggcctcaaacacacttcagagagaatttctgggcactttcagaaggctggtcactacagcacaaaagaagtcaaagttatcgcgttagtgctcccaaatctgaggcacagtttggagatgggggctctgagtcaaggggcaggctggggactgttccggtgcctgtgactatgggactgagtcagtcacagcccctctcattttcaccagtgaaggacataatgtatacagtgttccatttccaagacaaagtgccttaaattggcttaggtcagcagactacagaagaaacaggatattcgaggcccctgcttggatagctgatgcctgcttgtaggcctccccctttccccgcttaccccccattagttgccttcaccagaaccgaagaagtttagtctaaaatgaaagcttactactagcctgcaaaatagctccctttgtctgttcttatcagccagcccagctacttaggtcgtaagtgaaatacttgaagagcccctgagctgactaggattgcaatgcattgtgggctgtgacaaaatgcagcaagacaaccctaaaaacaaacaaacaaacaaaaacacctaaagcccctgcctaacaatcaataggtgacgtccaggaaggttgtgaccccatagtactcagcctgtgggaaactgggggaggagcctgcacactagtggacaaattgcttgttgaaactgctgggtgtgcctgtccgtcagacacccgattttgcaagactgactgtttattaaaaat

SEQ ID NO: 8 - DNA, 16 bp, synthetic construct
  source 1..16: mol_type = other DNA
gtcacgagctcttaag

SEQ ID NO: 9 - DNA, 16 bp, synthetic construct
  source 1..16: mol_type = other DNA
gtaggtagaatggtct

SEQ ID NO: 10 - DNA, 16 bp, synthetic construct
  source 1..16: mol_type = other DNA
gtaggtagaatggtct

SEQ ID NO: 11 - DNA, 16 bp, synthetic construct
  source 1..16: mol_type = other DNA
gtcacgagctcttaag

SEQ ID NO: 12 - DNA, 16 bp, synthetic construct
  source 1..16: mol_type = other DNA
gtagtgagtgtcacga

SEQ ID NO: 13 - RNA, 19 nt, synthetic construct
  misc_feature 1..19: note = siRNA
  source 1..19: mol_type = other RNA
ctattatagtcgtcctcaa

SEQ ID NO: 14 - RNA, 19 nt, synthetic construct
  misc_feature 1..19: note = siRNA
  source 1..19: mol_type = other RNA
gagaagcaggagttggagt

SEQ ID NO: 15 - RNA, 20 nt, synthetic construct
  source 1..20: mol_type = other RNA
gatggtcttgtgtctggact

SEQ ID NO: 16 - RNA, 20 nt, synthetic construct
  source 1..20: mol_type = other RNA
aatcagggaagagcatgagc

SEQ ID NO: 17 - RNA, 20 nt, synthetic construct
  source 1..20: mol_type = other RNA
cttgcagtggcccttggcat

SEQ ID NO: 18 - RNA, 20 nt, synthetic construct
  source 1..20: mol_type = other RNA
ctatgccaagggccactgca

SEQ ID NO: 19 - DNA, 15 bp, synthetic construct
  source 1..15: mol_type = other DNA
aacacgtctatacgc